SEQUENCE LISTING (22 sequences)

SEQ ID NO: 1 - protein, 447 aa, synthetic construct
  REGION 1..447: note = Synthetic Construct
EVQLVESGGGVVQPGRSLRLSCAASGFTFSRFGMHWVRQAPGKGLEWVAFISNDGSNKYYADSVKGRFTISRDNSKNTLYLQMNSLRPEDTAVYYCARGRAYGSGSLFDPWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 2 - protein, 214 aa, synthetic construct
  REGION 1..214: note = Synthetic Construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
  REGION 1..447: note = Synthetic Construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 219 aa, synthetic construct
  REGION 1..219: note = Synthetic Construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 114 aa, synthetic construct
  REGION 1..114: note = Synthetic Construct
QSVEESGGRLVTPGTPLTLTCTVSGFSLSTYAMIWVRQAPGKGLEYIGFIDNDYKAFYATWTNGRFTVSRTSTTVDLKMTSLTTEDTATYFCARYGSSSASDLWGQGTLVTVSS

SEQ ID NO: 6 - protein, 110 aa, synthetic construct
  REGION 1..110: note = Synthetic Construct
ALVMTQTPASVSAAVGDTVTIKCQASESIITWLAWYQQRPGQPPKLLIYRASTLASGVPSRFKGSGSGTQFTLTISDLEAADAAIYYCQNNAGDSSYGFAFGGGTEVVVK

SEQ ID NO: 7 - protein, 113 aa, synthetic construct
  REGION 1..113: note = Synthetic Construct
QSVEESGGRLVTPGTPLTLTCTVSGIDLSGSAMSWVRQAPGKGLEWIGIIYARGGTYYATWAQGRFTISKTSTTVDLKITSPTIEDTATYFCSRGYTDGFDLWGQGTLVTVSS

SEQ ID NO: 8 - protein, 111 aa, synthetic construct
  REGION 1..111: note = Synthetic Construct
ALVMTQTPSPVSAAVGGTVSINCQSTKSIYNNKYLSWYQQKPGQPPKLLIYDASDLASGVPSRFKGSGSGTQFTLTISGVQADDAATYYCLGGYSSDSENAFGGGTEVVVK

SEQ ID NO: 9 - protein, 110 aa, synthetic construct
  REGION 1..110: note = Synthetic Construct
ALVMTQTPASVSEPVGGTVTIKCQASQSISSWLAWYQQKPGQPPKLLIYRASTLASGVSSRFKGSGSGTDFTLTISDLEAADAATYYCQTSGAMGTYGGAFGGGTEVVVK

SEQ ID NO: 10 - protein, 113 aa, synthetic construct
  REGION 1..113: note = Synthetic Construct
QSVEESGGRLVTPGTPLTLTCTASGFSLSSYYMSWVRQAPGKGLEWIGFIYTDGSTYYASWAKGRFTISKTSTTVDLKITSPTTEDTATYFCARYSGSGLDLWGLGTLVTVSS

SEQ ID NO: 11 - protein, 120 aa, synthetic construct
  REGION 1..120: note = Synthetic Construct
QSLEESGGDLVKPGASMTLTCTASGFSFSSGYWICWVRQAPGKGLEWIACIHSVRSHMTYYASWAKGRFTISKTSSTTVTLQMTSLTAAATATYFCARDASGVWNYFTLWGPGTLVTVSS

SEQ ID NO: 12 - protein, 108 aa, synthetic construct
  REGION 1..108: note = Synthetic Construct
AEVVMTQTPSSVSAAVGGTVTIKCQASQNINSWLSWYQQKPGQRPKLLIYSASTLASGVPSRFEGSGSGTEYTLTISDLECDDAATYYCQSSYGSSYDFGGGTEVVVK

SEQ ID NO: 13 - protein, 124 aa, synthetic construct
  REGION 1..124: note = Synthetic Construct
QEQLVESGGGLVQPEGALTLTCTASGLDFSSSYWICWVRQAPGKGLEWIACVHAGSGGYNYYATWAKGRFTISRTSSTTVTLQMTSLTAADTATYFCARGVFPDYVDATLFNLWGPGTLVTVSS

SEQ ID NO: 14 - protein, 110 aa, synthetic construct
  REGION 1..110: note = Synthetic Construct
AAVMTQTASPVSAAVGGTVTINCQASQSVSSAYLSWYQQKPGQPPKLLIYKASTLASGVSSRFKGSGSGTEYTLTISGVQCDDAATYYCLYGDYSGRSNAFGGGTEVVVK

SEQ ID NO: 15 - protein, 121 aa, synthetic construct
  REGION 1..121: note = Synthetic Construct
QEHMEESGGDLVKPEGSLTLTCTASGFSFSRMYWICWVRQAPGKGLEWIACIYTGDGNTYYASWAKGRFTISKTSSSTVTLQMTSLTAADTATYFCARDPDGYSIYYFNLWGPGTLVTVSS

SEQ ID NO: 16 - protein, 110 aa, synthetic construct
  REGION 1..110: note = Synthetic Construct
ALVMTQTPASVSTAVGGTVTISCQASQSINSWLAWYQQKPGQRPNLLIYGASKLPSGVPSRFKGSGSGTEFTLTISDLECSDAATYYCAGYKTYSNDDNAFGGGTEVVVK

SEQ ID NO: 17 - protein, 119 aa, synthetic construct
  REGION 1..119: note = Synthetic Construct
QQLVESGGGLVQPGASLTLTCTASGFSFSSSYWICWVRQAPGKGLEWSACIYGGIPYYASWAKGRFTISKTSSTTVTLQMTSLTVADTATYFCARDDYYKSAWGGYNLWGPGTLVTVSS

SEQ ID NO: 18 - protein, 112 aa, synthetic construct
  REGION 1..112: note = Synthetic Construct
AAVMTQTPSPVSAAVGGTVTISCQASQSVYGNNYFAWFQQKPGQPPKLLIYKASTLASGVPSRFKGSGSGTQFTLTISGVECDDAATYYCAGYKSYSNDGYAFGGGTEVVVK

SEQ ID NO: 19 - protein, 121 aa, synthetic construct
  REGION 1..121: note = Synthetic Construct
EVQLVESGGGVVQPGRSLRLSCAASGFTFSRFGMHWVRQAPGKGLEWVAFISNDGSNKYYADSVKGRFTISRDNSKNTLYLQMNSLRPEDTAVYYCARGRAYGSGSLFDPWGQGTLVTVSS

SEQ ID NO: 20 - protein, 107 aa, synthetic construct
  REGION 1..107: note = Synthetic Construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIK

SEQ ID NO: 21 - protein, 121 aa, synthetic construct
  REGION 1..121: note = Synthetic Construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSS

SEQ ID NO: 22 - protein, 112 aa, synthetic construct
  REGION 1..112: note = Synthetic Construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVK

(19) United States
(12) Patent Application Publication
(10) Pub. No.: US 2025/0304709 A1
(43) Pub. Date: Oct. 2, 2025

(54) MULTISPECIFIC BINDING MOLECULES AND METHODS OF USE THEREOF

(71) Applicant: Eli Lilly and Company, Indianapolis, IN (US)

(72) Inventors: Riazul ALAM, Fishers, IN (US); Johnny Eugene CROY, Fishers, IN (US); Hiroaki TANI, Indianapolis, IN (US)

(21) Appl. No.: 18/859,878

(22) PCT Filed: Apr. 28, 2023
(86) PCT No.: PCT/US2023/066336

§ 371 Date: Oct. 24, 2024

Related U.S. Application Data
(60) Provisional application No. 63/338,480, filed on May 5, 2022.

(51) Int. Cl. C07K 16/28; A61K 39/00; A61K 47/68

(52) U.S. Cl. CPC C07K 16/2881; A61K 47/6879; C07K 16/2803; A61K 2039/505; A61K 2039/54; C07K 2317/31; C07K 2317/60; C07K 2317/92

(57) ABSTRACT

Multispecific binding molecules having a first binding domain targeting a blood brain barrier target and a second binding domain targeting a neuron target, astrocyte target and/or glial cell target, and uses thereof in aiding the treatment of central nervous system diseases including neurodegenerative diseases such as Alzheimer's disease, Huntington's disease, Parkinson's diseases, Progressive Supranuclear Palsy (PSP), Amyotrophic Lateral Sclerosis (ALS), Frontal Temporal Dementia (FTD), autism, catalepsy, encephalitis, migraine, and Tourette's.

### CROSS-REFERENCE TO RELATED APPLICATIONS

This patent application claims the benefit of U.S. Provisional Patent Application Ser. No. 63/338,480, filed May 5, 2022, which is hereby incorporated by reference in its entirety.

### REFERENCE TO A SEQUENCE LISTING

The instant application contains a Sequence Listing which has been submitted electronically in ST.26 XML format and is hereby incorporated by reference in its entirety. Said ST.26 XML Sequence Listing was created on Apr. 4, 2023, is named 22892.xml and is 31,460 bytes in size. No new matter is added.

### FIELD OF THE INVENTION

The present invention is in the field of medicine, particularly in the field of treatment of central nervous system (CNS) diseases. More specifically, the present disclosure relates to compositions comprising a multispecific binding molecule having a first binding domain targeting blood brain barrier targets and a second binding domain targeting neuron targets, astrocyte targets and/or glial cell targets (collectively, the CNS Targets), and uses thereof, in treating CNS diseases. The multispecific binding molecules of the present disclosure aide in the treatment of CNS diseases by enhancing passage of therapeutics across the blood brain barrier and delivery thereof to intended CNS Targets.

### BACKGROUND OF THE INVENTION

The CNS is arguably the most complex and highly organized organ system of the human body. The CNS consists of two major organs, the brain and spinal cord, and is composed of both glial cells and neurons. In vertebrates such as humans, neurons are classified into three types: sensory neurons and motor neurons, parts of which may span both the CNS and peripheral nervous system, and interneurons which are thought to be almost exclusively within the CNS.

Diseases of the CNS are disorders that affect the structure and/or functions of the brain and/or spinal cord. CNS diseases or disorders can include autoimmune disorders including neuromyelitis optica, multiple sclerosis, and anti-myelin oligodendrocyte glycoprotein; oncology disorders including astrocytomas, oligodendrogliomas, glioblastomas and other CNS tumors; neurodegenerative disorders including Alzheimer's disease, Huntington's disease, Parkinson's diseases, Progressive Supranuclear Palsy (PSP), Amyotrophic Lateral Sclerosis (ALS) and Frontal Temporal Dementia (FTD); as well as other CNS disorders such as autism, catalepsy, encephalitis, migraine, and Tourette's.

CNS diseases have traditionally proven very difficult to treat. One reason is that a therapeutic must traverse the blood brain barrier to enter the CNS. The blood brain barrier is a specialized, semipermeable border of endothelial cells that selectively prevents solutes, including pathogens, from passing into the CNS. The blood brain barrier allows for the passage of some molecules via passive diffusion and engages in selective, active transport of some molecules crucial to neural function. In addition to the challenges posed by the blood brain barrier, once a therapeutic traverses the blood brain barrier, the therapeutic must still act on its intended target within the CNS. Some studies have estimated that less than 1% of some therapeutics cross the blood brain barrier.

Blood brain barrier shuttles, or chaperones, for improving passage of the therapeutics across the blood brain barrier and into the CNS have been described for over two decades. For example, WO2003/009815 (US2003/0129186) describes the use of antibodies directed to transferrin receptors (“TFR”) for modulating blood brain barrier transport. However, attempts at using anti-TFR antibodies as conjugates to shuttle therapeutics across the blood brain barrier have proven both challenging and insufficient in significantly improving the treatment of CNS disease (see, for example, Syvanen S, Hultqvist G, Gustavsson T, et al. Efficient clearance of Abeta protofibrils in AbetaPP-transgenic mice treated with a brain-penetrating bifunctional antibody. Alzheinmr's Res Tiher. 2018; 10:49; Couch J A, Yu Y J, Zhang Y, et al. Addressing safety liabilities of TfR bispecific antibodies that cross the blood-brain barrier. Sci Trans Med. 2013; 5:183ra57, discussing pharmacokinetic, toxicity and off-target issues associated with targeting TfR as a blood brain barrier shuttle).

Thus, there remains a need for enhancing treatment of CNS diseases that overcomes the challenges described above. More particularly, there exists a need for improving the delivery of therapeutics across the blood brain barrier and providing enhanced selective targeting of CNS Targets within the CNS. Such enhancements must be able to deliver therapeutics across the blood brain barrier with increased efficiency and improve delivery to specific CNS Targets within the CNS such that the shuttled therapeutic is delivered to its intended therapeutic target within the CNS. Such enhancements should not be more invasive than the therapeutic molecule, enable peripheral systemic delivery of the therapeutic to overcome the need to invasively deliver the therapeutic directly into the CNS, should not be attendant upon unacceptable immunogenicity and off-target issues, and should demonstrate acceptable stability and developability. Such enhancements should also not interfere with the properties of the therapeutic necessary for treating the CNS disease.

The present disclosure provides multispecific binding molecules for enhancing treatment of CNS diseases. More particularly, the present disclosure provides multispecific binding molecules having a first binding domain that specifically binds a blood brain barrier target (a “BBB Target”) and a second binding domain that specifically binds a neuron target, an astrocyte target and/or a glial cell target (a “CNS Target”) thereby enhancing delivery of a therapeutic composition to its intended therapeutic target. Such multispecific binding molecules are capable of being linked to a therapeutic composition for aiding the treatment of CNS diseases by enhancing passage of the therapeutic across the blood brain barrier and delivery to specific CNS Targets within the CNS such as a neuron target, an astrocyte target and/or a glial cell target, whereby the therapeutic composition modulates its intended therapeutic target.

### SUMMARY OF THE INVENTION

Embodiments of the present disclosure comprise a multispecific binding molecule comprising a first binding domain and a second binding domain, wherein the first binding domain specifically binds a blood brain barrier target and the second binding domain specifically binds at least one of a neuron target, an astrocyte target and/or a glial cell target or another parenchymal target in the CNS. According to some embodiments, the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A. According to some embodiments, the neuron target is one of L1CAM, THY1, KIT, GRIA1, GRM2, TMEM130, NSG1, BASP1, NSTR1, SLC22A17, NCAM1, NCAM2, SLC6A17, SCN3B and P2Y12R. According to some embodiments in particular, the blood brain barrier target is TFR and the neuron target is L1 CAM.

In some embodiments, multispecific binding molecules of the present disclosure comprise a bispecific antibody, heterodimeric immunoglobulin antibody, or fragment thereof. Additionally, in some embodiments of multispecific binding molecules of the present disclosure, the first binding domain is a Fab, scFv, Fv, or scFab. According to some embodiments, the second binding domain is a Fab, scFv, Fv, or scFab. Additionally, in some embodiments the first binding domain and the second binding domain are linked by one of a thiol-based reactive group, amine-based reactive group, aldehyde-reactive groups or carbohydrate-based groups. In additional embodiments, the first binding domain and the second binding domain are linked by one of a PEG-linker, an amino acid linker, and a glycan linker.

In further embodiments of the multispecific binding molecules of the present disclosure, a therapeutic composition is linked to the multispecific binding molecule. According to some embodiments, the therapeutic composition is one of a peptide, antibody or fragment thereof, or an oligonucleotide. In some such embodiments, the therapeutic composition targets include but are not limited to MAPT, SNCA, APP, BAC1, ATXN2, ATXN3, SARM1, APOE, FMR1, LRRK2, HTT, SOD1, SCN10A, SCN9A or CACNAlB.

Additionally, in some embodiments, multispecific binding molecules of the present disclosure are linked to a carrier molecule.

Further embodiments of the present disclosure include pharmaceutical compositions comprising a multispecific binding molecule of the present disclosure and one or more pharmaceutically acceptable carriers, diluents, or excipients.

Embodiments of the present disclosure also include a DNA molecule comprising a polynucleotide sequence encoding a polypeptide chain comprising one of a HC, LC, HCVR or LCVR of a multispecific binding molecule of the present disclosure.

According to some embodiments, a method of treating a central nervous system disease comprising administering to a patient in need thereof a therapeutically effective amount of a multispecific binding molecule of the present disclosure is provided. Embodiments of the present disclosure also include a method of treating a central nervous system disease comprising administering a therapeutic composition to a patient in need thereof, wherein the therapeutic composition is linked to a multispecific binding molecule of the present disclosure, wherein the therapeutic composition accumulation in peripheral tissue is reduced compared to the therapeutic composition when not linked to the multispecific binding molecule. According to some embodiments, at least 24 hours, 48 hours, 72 hours, 96 hours, 120 hours, or 144 hours after the step of administering, the therapeutic composition accumulation in peripheral tissue is reduced compared to the therapeutic composition when not linked to the multispecific binding molecule.

According to some embodiments, the present disclosure provides a method of delivering a therapeutic composition to a CNS Target within the CNS comprising administering the therapeutic composition to a patient in need thereof, wherein the therapeutic composition is linked to a multispecific binding protein of the present disclosure, wherein the therapeutic composition demonstrates reduced accumulation in peripheral tissue of the patient. In some such embodiments, the therapeutic composition demonstrates reduced accumulation in bone marrow of the patient. According to some embodiments, the CNS Target is one of a neuron target, an astrocyte target and a glial cell target. According to some embodiments, the central nervous system disease is a neurodegenerative disorder. According to some embodiments, the central nervous system disease is Alzheimer's disease Huntington's disease, Parkinson's diseases, Progressive Supranuclear Palsy (PSP), Frontal Temporal Dementia (FTD), autism, catalepsy, encephalitis, migraine, and Tourette's.

Embodiments of the present disclosure include a multispecific binding molecule of the present disclosure for use in therapy. Embodiments also include a multispecific binding molecule of the present disclosure, for use in the treatment of a central nervous system disease. According to some such embodiments, the CNS disease is Alzheimer's disease, Huntington's disease, Parkinson's diseases, Progressive Supranuclear Palsy (PSP), Amyotrophic Lateral Sclerosis (ALS), Frontal Temporal Dementia (FTD), autism, catalepsy, encephalitis, migraine, and Tourette's. Further, the present disclosure in some embodiments provides a multispecific binding molecule of the present disclosure for use in the manufacture of a medicament for use in the treatment of a CNS disease.

### BRIEF DESCRIPTION OF THE DRAWINGS

FIG. 1 is a schematic of a multispecific binding molecule in the form of a heterodimeric IgG antibody of the present disclosure.

FIG. 2 is a schematic of a multispecific binding molecule in the form of a heterodimeric IgG antibody having a first binding domain that binds a blood brain barrier target, TFR, and a second binding domain that binds a neuron target, L1CAM, of the present disclosure.

FIG. 3 shows high content imaging data demonstrating cellular activity (binding, internalization, and degradation properties) of exemplified multispecific binding molecule of the present disclosure.

FIGS. 4A and 4B show brain pharmacokinetic data for exemplified multispecific binding molecule of the present disclosure.

FIG. 5A through 5E show in vivo pharmacodynamic data for exemplified multispecific binding molecule of the present disclosure.

FIG. 6A through 6J show in vivo brain and peripheral accumulation of exemplified multispecific binding molecule of the present disclosure.

### DETAILED DESCRIPTION OF THE INVENTION

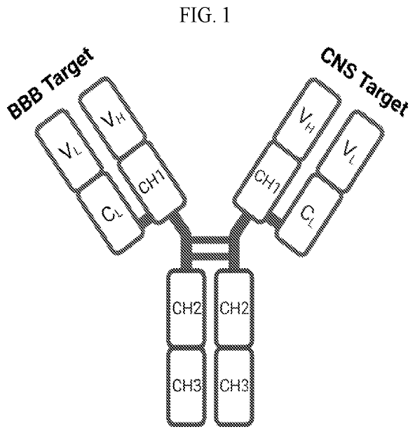

The term “multispecific”, as used herein, refers to a molecule or compound that comprises two or more distinct binding domains. A multispecific binding molecule, as described herein, can bind two or more different targets; a blood brain barrier target and a CNS Target. Embodiments of multispecific binding molecules of the present disclosure may include bispecific antibodies and/or fragments of antibodies, such as Fab, scFv, Fv, or scFab, wherein portions of the antibody or fragments thereof form binding domains of the molecule such that the multispecific binding molecule may bind a blood brain barrier target and a CNS Target, as described herein. Embodiments of multispecific binding molecules include two scFv linked together, for example via a peptide linker (e.g., GS linker), wherein one scFv forms a binding domain for a blood brain barrier target and the other scFv forms a binding domain for a CNS Target. Multispecific binding molecules of the present disclosure also include a VHH linked to one of more fragments of an antibody. Other multispecific binding molecules of the present disclosure include heterodimeric immunoglobulin G (IgG) antibodies, as exemplified in FIG. 1, wherein the amino terminus (N-term) of each “arm”, comprising a HCVR/LCVR pair, form a binding domain exhibiting selective monovalent binding to its cognate antigen (e.g., the binding domain of one “arm” specifically binds a blood brain barrier target and the binding domain of the other “arm” specifically binds a CNS Target).

The term “binding domain” refers to a portion of a multispecific binding molecule of the present disclosure that binds a target (e.g., a BBB Target or a CNS Target, as exemplified in FIG. 1). For example, a binding domain of a multispecific binding molecule may bind a blood brain barrier target, such as TFR. Likewise, a different binding domain of a multispecific binding molecule of the present disclosure may bind a CNS Target, such as L1CAM. The terms “bind” and “binds” as used herein are intended to mean, unless indicated otherwise, the ability of a binding domain of a multispecific binding molecule to form a chemical bond or attractive interaction with another protein or molecule, which results in proximity of the two proteins or molecules as determined by common methods known in the art.

Blood brain barrier target, as used herein refers to membrane receptors that are presented on the apical side of the endothelial blood-brain barrier and actively aid in, facilitate and/or promote the internalization of molecules external to the CNS (e.g., in serum) into the CNS. Blood brain barrier targets, according to the present disclosure, include but are not limited to TFR, Low Density Lipoprotein Receptor-related Protein 1 (LRP1), LRP2 or megalin/GP330, LRP3, LRP5, LRP6, LRP8, insulin-like growth factor receptor 1 (IGF1R), Transferrin Receptor-1 (or TFR/CD71), CD98 heavy chain (SLC3A2), large amino acid transporter small subunit 1 (LAT-1), insulin receptor, low-density lipoprotein (LDL) receptor, CDC50A and the like.

CNS Targets, as used herein, refers to antigens present on the outer membrane of one of a neuron, astrocyte and/or glial cell as well as other parenchymal antigens in the CNS. Multispecific binding molecules of the present disclosure comprise a binding domain targeting a CNS Target, facilitating bringing a therapeutic composition linked to the multispecific binding molecule in proximity of a desired CNS Target whereby the therapeutic composition may act on its intended therapeutic target. Antigens, including single transmembrane receptors, multipass membrane protein receptors and cell surface proteoglycans, present on the outer membrane of neurons include, but are not limited to, L1CAM, THY1, KIT, GRIA1, GRM1, GRM2, TMEM130, NSG1, BASP1, NSTR1, SLC22A17, NCAM1, NCAM2, SLC6A17, SCN3B, P2Y12R, CADM1, PARM1, NSG1 and KIDINS220. Antigens, including single transmembrane receptors, multipass membrane protein receptors and cell surface proteoglycans, present on the outer membrane of microglia cells include, but are not limited to, TREM2, TMEM119, CD1 Ib, CD14, CD16, CD32, CD33, CD40, CD45, CD64, CD68, CD80, CD115, CD172a, CX3CR1, FCER1G, F4/80, FCRLS, Siglec, Glut5, and P2Y12. Antigens, including single transmembrane receptors, multipass membrane protein receptors and cell surface proteoglycans, present on the outer membrane of astrocytes include but are not limited to CD49f, AQP4, GLT1, GLAST, A2AR, TGFbR, S1P1, IL1R<IL6R, IL17R and IFNgR.

The region of a target, bound by a binding domain of a multispecific binding molecule, may be a linear or conformational region of the target. The region of a target, bound by a binding domain of a multispecific binding molecule, may be determined according to different experimental techniques. It is understood that the determination of a region of a target bound by a multispecific binding molecule may vary based on the different mapping techniques used and may also vary with the different experimental conditions used, for example, due to the conformational changes or cleavages of the target induced by specific experimental conditions. Binding mapping techniques are known in the art (e.g., Rockberg & Nilvebrant, Epitope Mapping Protocols: Methods in Molecular Biology, Humana Press, 3rd ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

The term “specific for”, as used herein in relation to a binding domain, refers to the ability of a binding domain of a multispecific binding molecule of the present disclosure to bind, associate with and/or modulate a particular target molecule, over background levels of non-target levels. In some embodiments, “specific for” may be demonstrated by affinity (Kd) measures for the target over non-targets.

The term, “modulate” or “modulates” and the like, as used herein, refers to altering or changing a measurable value and includes both altering or changing such a measurable value upwards (i.e., upmodulate or upmodulating) or downwards (i.e., downmodulate or downmodulating).

Multispecific binding molecules of the present disclosure may also be linked to one or more therapeutic compositions, such as a therapeutic antibody, or fragment thereof, peptide, oligonucleotide, small molecule, nanoparticle, lipid nanoparticle, exosome, or a combination thereof (it is also understood that the multispecific binding molecules, or therapeutic, may be fully or partially encapsulated by a lipid nanoparticle or exosome). Exemplary therapeutic oligonucleotides include antisense oligonucleotide, RNA interference, RNA activation, and guide RNA-directed target RNA editing using systems such as CRISPR-Cas9 and ARCUS Nuclease systems. Exemplary therapeutic oligonucleotides, antibodies or fragments thereof, and/or peptides which are known in the field and are directed to a therapeutic target gene or protein within the CNS (i.e., a “therapeutic target” that is distinct from the blood brain barrier target and CNS Target) that is associated with a protein involved in a disease of the CNS, such as beta-amyloid, BACE1, MAPT, SNCA, ATXN2, ATXN3, SARM1, APOE, FMR1, LRRK2, HTT, SOD1, SCN10A, SCN9A, CACNAlB and the like, modulation of which is intended to bring about a therapeutic effect, may be linked to multispecific binding molecules of the present disclosure.

Furthermore, therapeutic compositions may be linked to multispecific binding molecules of the present disclosure in a variety of ways and at various positions or portions of the multispecific binding molecule such that such linkage does not interfere with the binding of the multispecific binding molecule to the blood brain barrier and CNS Target and does not interfere with the therapeutic properties of the therapeutic composition when linked.

The term linker, as used herein, refers to an atom, group of atoms, molecule or compound (such as an amino acid or group of amino acids) comprising at least one bond that attaches or links a molecule or compound to another molecule or compound. Linkers may be comprised of varying compositions including polyethylene glycol (PEG) units, straight, branched and aromatic carbon-based units, amino-acids and glycans. Linkers, according to the present disclosure, may also include hydrolysable and/or enzymatically degradable linkers. Linkers may also attach multiple therapeutic compositions to a multispecific binding molecule of the present disclosure.

The terms “linked” and “conjugated”, as used interchangeably herein, refers to a first molecule or compound, for example a polypeptide, being associated, attached, connected or otherwise joined to a second molecule or compound, for example a nucleotide (or polynucleotide) or polypeptide. For example, a polypeptide can be linked to a polynucleotide sequence. Likewise, a peptide sequence can be linked to a second peptide sequence via covalent or non-covalent interactions to form a multimeric peptide. In some embodiments of the present disclosure, an antibody or fragment thereof is linked to an oligonucleotide. The term, “conjugated peptide” or “peptide conjugate,” as used herein, refers to a peptide or protein that is covalently (including reversibly-covalent) or non-covalently linked with another molecule, moiety, compound or group.

Particular embodiments of the present disclosure includes a heterodimeric IgG multispecific binding molecule in which one arm specifically binds a blood brain barrier target, for example TFR-1, and the other arm specifically binds a CNS Target, for example a neuron target such as L1CAM, and the IgG multispecific binding molecule is linked with, for example by way of a chemically functionalized linker, an siRNA therapeutic composition, that provides covalent attachment to the multispecific binding molecule scaffold. Examples of chemically functionalized linkers comprise thiol-based reactive groups (e.g., maleimides, sulfhydryls, haloacetyls, thiosulfonates, disulfide), amine-based groups (e.g., N-hydroxysuccimide esters, imidoesters, etc) and aldehyde-reactive groups (e.g., hydrazides and alkoxyamines), and carbohydrate-based groups (e.g., native Fc glycan, engineered glycans etc.).

The term, “amino acid with a functional group available for conjugation,” as used herein, refers to any natural or unnatural amino acid, or derivative thereof, with a functional group that may be conjugated to a conjugate moiety directly or by way of, for example, a conjugate linker. Examples of such functional groups include, but are not limited to, alkynyl, alkenyl, amino, azido, bromo, carboxyl, chloro, iodo and thiol groups. Likewise, examples of natural amino acids including such functional groups include C (thiol), D (carboxyl), E (carboxyl), K (amino) and Q (amide).

The multispecific binding molecules of the present disclosure can be used in aiding in the treatment of patients, particularly for aiding in treatment of CNS diseases, including neurodegenerative, oncology and autoimmune disorders of the CNS. When used in reference to the multispecific binding molecules of the present disclosure, “treatment” and/or “treating” and/or “treat” refer to the use of multispecific binding molecules of the present disclosure in conjunction with a therapeutic composition (e.g., the therapeutic composition linked or conjugated to the multispecific binding molecule), whereby the multispecific binding molecule aides in delivering the therapeutic composition across the blood brain barrier and to a CNS Target, whereby the therapeutic composition is able to modulate its intended therapeutic target, thereby fully or partially: slowing, interrupting, arresting, controlling, stopping, or reversing the progression of a CNS disorder, but does not necessarily indicate a total elimination of all disorder symptoms. Treatment includes administration of a multispecific binding molecule of the present disclosure comprising a therapeutic composition linked or conjugated thereto for treatment of a CNS disease or condition in a human that would benefit from delivering the therapeutic composition across the blood brain barrier and to a CNS Target in order to aid the therapeutic composition in bringing about a desired therapeutic effect.

As used interchangeably herein, the term “patient,” “subject,” and “individual,” refers to a human. In certain embodiments, the patient is further characterized with a CNS disease, disorder, or condition (for example, a CNS neurodegenerative disorder). In some embodiments, the patient may be further characterized as being at risk of developing a CNS disorder, disease, or condition.

Embodiments of multispecific binding molecules of the present disclosure include antibodies, or fragments thereof. As used herein, “antibody” includes immunoglobulin, or immunoglobulin-like molecules that binds an antigen and that comprise four polypeptide chains: two heavy chains (HC) and two light chains (LC) that are cross-linked via inter-chain disulfide bonds (as illustrated above). Each HC is comprised of a heavy chain variable region (VH) and a heavy chain constant region. Each LC is comprised of a light chain variable region (VL) and a light chain constant region. The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). Exemplary embodiments of antibody fragments or antigen-binding fragments, as used herein, comprise at least a portion of an antibody retaining the ability to specifically interact with an antigen or an epitope of the antigen, such as Fab, Fab′, F(ab′)₂, Fv fragments, scFv antibody fragments, scFab, disulfide-linked Fvs (sdFv), a Fd fragment.

### EXAMPLES

### Exemplified Multispecific Binding Molecule

Exemplified multispecific binding molecules of the present disclosure comprise a first binding domain specific for a blood brain barrier target and a second binding domain specific for a CNS Target. According to particular exemplified multispecific binding molecules of the present disclosure, the first binding domain binds TFR and the second binding domain binds L1CAM. Exemplified binding domains of multispecific binding molecules of the present disclosure comprise a heavy chain variable region (HCVR) and a light chain variable region (LCVR), which each comprise three complementarity determining regions (HCDRs 1-3 and LCDRs 1-3, respectively). Exemplified binding domains are set forth in Table 1.

TABLE 1
Exemplified Binding Domain Regions

| Binding | Target | | |
|---|---|---|---|
| Domain | (BBB) | HC Region | LC Region |
| BD1 | TFR | HC | LC |
| | | SEQ ID NO: 3 | SEQ ID NO: 4 |
| | | HCVR | LCVR |
| | | SEQ ID NO: 21 | SEQ ID NO: 22 |
| BD2 | TFR | HCVR | LCVR |
| | | SEQ ID NO: 5 | SEQ ID NO: 6 |
| BD3 | TFR | HCVR | LCVR |
| | | SEQ ID NO: 7 | SEQ ID NO: 8 |
| BD4 | TFR | HCVR | LCVR |
| | | SEQ ID NO: 9 | SEQ ID NO: 10 |
| BD5 | CD98 | HCVR | LCVR |
| | | SEQ ID NO: 11 | SEQ ID NO: 12 |
| BD6 | CD98 | HCVR | LCVR |
| | | SEQ ID NO: 13 | SEQ ID NO: 14 |
| BD7 | CD98 | HCVR | LCVR |
| | | SEQ ID NO: 15 | SEQ ID NO: 16 |
| BD8 | CD98 | HCVR | LCVR |
| | | SEQ ID NO: 17 | SEQ ID NO: 18 |
| | CNS | | |
| | Target | HC Region | LC Region |
| BD9 | L1CAM | HC | LC |
| | | SEQ ID NO: 1 | SEQ ID NO: 2 |
| | | HCVR | LCVR |
| | | SEQ ID NO: 19 | SEQ ID NO: 20 |

### Expression of Exemplified Multispecific Binding Molecule

Exemplified multispecific binding molecules may be expressed and purified essentially as follows. A glutamine synthetase (GS) expression vector (or vectors) containing the DNA encoding a polypeptide chain comprising an HCVR and LCVR (or, for example an HC and LC), or fragments thereof, that comprise respective binding domains of the multispecific binding molecules of the present disclosure, are used to transfect a Chinese hamster cell line, CHO (GS knockout, clone 1D3), by electroporation. The expression vector encodes an SV Early (Simian Virus 40E) promoter and the gene for GS. Expression of GS allows for the biochemical synthesis of glutamine, an amino acid required by the CHO cells. Post-transfection, cells undergo bulk selection with 50 M L-methionine sulfoximine (MSX). The inhibition of GS by MSX is utilized to increase the stringency of selection. Cells with integration of the expression vector cDNA into transcriptionally active regions of the host cell genome can be selected against CHO wild type cells, which express an endogenous level of GS. Transfected pools are plated at low density to allow for close-to-clonal outgrowth of stable expressing cells. The masterwells are screened for multispecific binding molecule expression and then scaled up in serum-free, suspension cultures to be used for production. Clarified medium, into which the exemplified multispecific binding molecule has been secreted, is applied to a Protein A affinity column that has been equilibrated with a compatible buffer such as phosphate buffered saline (pH 7.4). The column is washed to remove nonspecific binding components. The bound multispecific binding molecule is eluted, for example, by pH gradient (such as 0.1 M sodium phosphate buffer pH 6.8 to 0.1 M sodium citrate buffer pH 2.5) and neutralized with Tris, pH 8 buffer. Multispecific binding molecule fractions are detected, such as by SDS-PAGE or analytical size-exclusion, and then are pooled. Soluble aggregate and multimers may be effectively removed by common techniques including size exclusion, hydrophobic interaction, ion exchange, or hydroxyapatite chromatography. The multispecific binding molecule is concentrated and/or sterile filtered using common techniques. The purity of the multispecific binding molecules after these chromatography steps is greater than 98.6% (monomer). The multispecific binding molecules may be immediately frozen at −70° C. or stored at 4° C. for several months.

### Exemplified TFR and L1CAM Multispecific Binding Molecule

An exemplified multispecific binding molecule of the present disclosure is provided, comprising a heterodimeric IgG antibody. Accordingly, the exemplified multispecific binding molecule comprises a first heavy chain (HC1) and light chain (LC1), wherein the N-term of the heavy and light chain comprise a blood brain barrier target, TFR, binding domain, and further comprises a second heavy chain (HC2) and light chain (LC2), wherein the N-term of the second heavy and light chain comprise a neuron target, L1CAM, binding domain. Each heavy and light chain comprise a variable region, a heavy chain variable region (HCVR) having three complementarity determining regions (HCDRs1-3), and a light chain variable region (LCVR) having three LCDRs (LCDRs 1-3), respectively. The amino acid sequence for the respective polypeptide chains, and regions thereof of the exemplified multispecific binding molecule is set forth in Table 2.

TABLE 2
Amino Acid Sequences Comprising Exemplified Multispecific
Binding Molecule (“MBM1”)

| Region | SEQ ID NO |
|---|---|
| Heavy chain 1 (HC1) | SEQ ID NO: 3 |
| Heavy chain variable region (HCVR1) | SEQ ID NO: 21 |
| Light chain 1 (LC1) | SEQ ID NO: 4 |
| Light chain variable region (LCVR1) | SEQ ID NO: 22 |
| Heavy chain 2 (HC2) | SEQ ID NO: 1 |
| Heavy chain variable region (HCVR2) | SEQ ID NO: 19 |
| Light chain 2 (LC2) | SEQ ID NO: 2 |
| Light chain variable region (LCVR2) | SEQ ID NO: 20 |

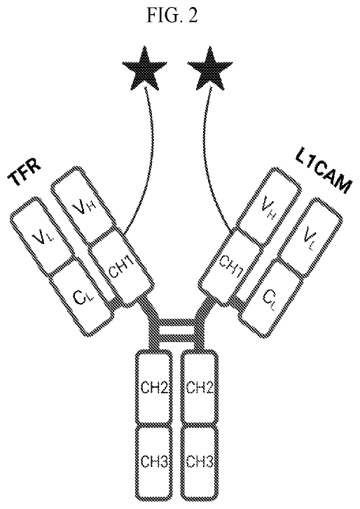

The exemplified multispecific binding molecule may be linked at the heavy or light chain constant region, for example CH1 of one or both HC1 and HC2, or for example the constant region of one or both LC1 and LC2 (for example, at a cysteine, either native or non-native engineered as known in the art, via a thiol-reactive maleimide-functionalized linker) to a therapeutic composition such as an siRNA therapeutic composition (represented in the below schematic by a star) targeting a therapeutic target as described above. An illustration of the exemplified multispecific binding molecule is provided in FIG. 2.

### Binding Affinity of Exemplified Multispecific Binding Molecules

Binding affinity and binding stoichiometry of the exemplified multispecific binding molecule to mouse TFR, and human and mouse L1CAM, is determined using a surface plasmon resonance assay on a Biacore T200 instrument primed with HBS-EP+(10 mM Hepes pH7.4+150 mM NaCl+3 mM EDTA+0.05% (w/v) surfactant P20) running buffer and analysis temperature set at 25° C. A human Fab capture kit (Cytiva P/N 28958325) is immobilized on a CM5 chip (Cytiva P/N 29104988) using standard NHS-EDC amine coupling on all four flow cells (Fc). Multispecific binding molecules (“MBM1” of Table 2, with or without conjugated siRNA (denoted by *) as described above) are prepared at 10 μg/mL by dilution into running buffer. Target (e.g., mouse TFR-mIgG1-Fc, mouse L1CAM-hIgG1-Fc and human L1CAM-hIgG1-Fc, respectively) are prepared at final concentrations of 100.0, 25.0, 6.25, 1.56, 0.39, 0.097, 0.024 and 0 (blank) nM by dilution into running buffer.

Each analysis cycle consists of (1) capturing multispecific binding molecule samples on separate flow cells (Fc2, Fc3 and Fc4); (2) injection of the respective concentration for the blood brain barrier target (e.g., TFR) over all Fc at 10 μL/min for 60 seconds followed by return to buffer flow for 1800 seconds to monitor dissociation phase; (3) injection of each human L1CAM concentration over all Fc at 100 μL/min for 120 seconds followed by return to buffer flow for 1200 seconds to monitor dissociation phase; (4) regeneration of chip surfaces with injection of 10 mM glycine, pH 1.5, for 30 seconds at 10 μL/min over all cells; and (5) equilibration of chip surfaces with a 10 μL (60-sec) injection of HBS-EP+. Data are processed using standard double-referencing and fit to a 1:1 binding model using Biacore T200 Evaluation software, version 2.0.3, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and presented in molar units. Results are provided in Table 3.

TABLE 3
Binding Affinity of Exemplified Binding
Domains to Respective Target at 25° C.

| Multispecific | | | | |
|---|---|---|---|---|
| Binding | | | | |
| Molecules | | | | |
| (*denotes | | | | |
| conjugated | | kₒₙ | k_{off} | K_{D} |
| siRNA) | Target | M⁻¹s⁻¹ | s⁻¹ (10⁻⁴) | pM |
| MBM1 | mTFR | 3.6E4 | 1.7E−4 | 4.9E−9 |
| MBM1 | mL1CAM | 4.0E5 | 2.0E−4 | 5.1E−10 |
| MBM1 | hL1CAM | 4.3E5 | 1.1E−4 | 2.7E−10 |
| MBM1* | mTFR | 9.2E4 | 9.6E−4 | 1.04E−8 |
| MBM1* | mL1CAM | 2.2E5 | 2.7E−4 | 1.2E−9 |
| MBM1* | hL1CAM | 2.6E5 | 2.3E−4 | 8.9E−10 |

These results demonstrate exemplified multispecific binding molecules of the present invention bind blood brain barrier target TFR and CNS Target (neuron target) L1CAM with high affinity at 25° C.

### Simultaneous Binding of TFR and L1CAM

A BIAcore T200 instrument is used to determine whether TFR and L1CAM can simultaneously bind to the exemplified multispecific binding molecule (MBM1), described in detail above. Except as noted, all reagents and materials are from Cytiva (Upsala, Sweden). All measurements are performed at 25° C. HBS-EP+ buffer (150 mM sodium chloride, 3 mM EDTA, 0.05% (w/v) surfactant P-20, and 10 mM HEPES, pH7.4) is used as the running buffer and sample buffer. A human Fab capture kit (Cytiva P/N 28958325) is immobilized on a CM5 chip (Cytiva P/N 29104988) using standard NHS-EDC amine coupling on all four flow cells (Fc) The exemplified multispecific binding molecule (diluted to 50 μg/ml) is first captured on flow cell 2 (with a 30 second injection at 30 μl/min, yielding 100 response units (RU) of multispecific binding molecule capture), followed by injection of human or mouse L1CAM at 500 nM for 5 min to saturate L1CAM binding site (a binding signal of 15 ARU is observed). Flow cell 1 is a blank only control. After binding of L1CAM (flow cell 2), mouse TFR at 500 nM is then injected for 5 min and additional binding signal (of 8 A RU) is observed. Chip surface is then regenerated using 10 mM Glycine pH 1.5. The same process is repeated except with a reverse order of mouse TFR first followed by human or mouse L1CAM. Results show an increase in response units (initial 10 RU from TFR and then additional 13 RU from L1CAM) from the two ligands binding to the multispecific binding molecule demonstrating the exemplified multispecific binding molecule of the present invention can bind human or mouse L1CAM and mouse TFR simultaneously.

### In Vitro Binding, Internalization and Degradation Assessment in Murine Cortical Neurons

Fluorescence signal corresponding to total levels, and internalization of, antibodies and binding molecule-siRNA conjugates is measured by performing a high content live cell imaging assay in primary mouse cortical neurons. Briefly, mouse primary cortical neurons are isolated from wild type C57BL6 mouse embryos at E18. Cells are plated in poly-D-lysine coated 96-well plates at a density of 40 k cells/well and cultured in NbActiv1 (BrainBits, LLC) containing 1% Antibiotic/Antimycotic (Coming) for 7 days at 37° C. in a tissue culture incubator in a humidified chamber with 5% CO2. On day 7, medium is removed from each well and replaced with culture media with 5 ug/ml (33 nM) of either: (i) hIgG4 isotype control antibody (Iso mAb), (ii) monovalent TFR antibody (having a single TFR targeting arm and a nonspecific arm (mvTFR), (iii) MBM1 (of Table 2), or the above molecules having an siRNA linked at HC Constant region 1 (denoted with *), together with 10 ug/ml (0.2 uM) of anti-human IgG Fcγ fragment specific Fab fragment (Jackson Immuno #109-007-008) labelled with either DyLight 650 (Thermo Fisher #62266), DL650 together with BHQ3 dye (BioSearch Tech BHQ-3000S-5) or pHAb dye (Promega #G9845) in culture media with 6.7 uM (1 mg/ml) goat gamma globulin (Jackson Immuno #005-000-002) and incubated overnight with live cells grown in a 96 well plate at 37 C.

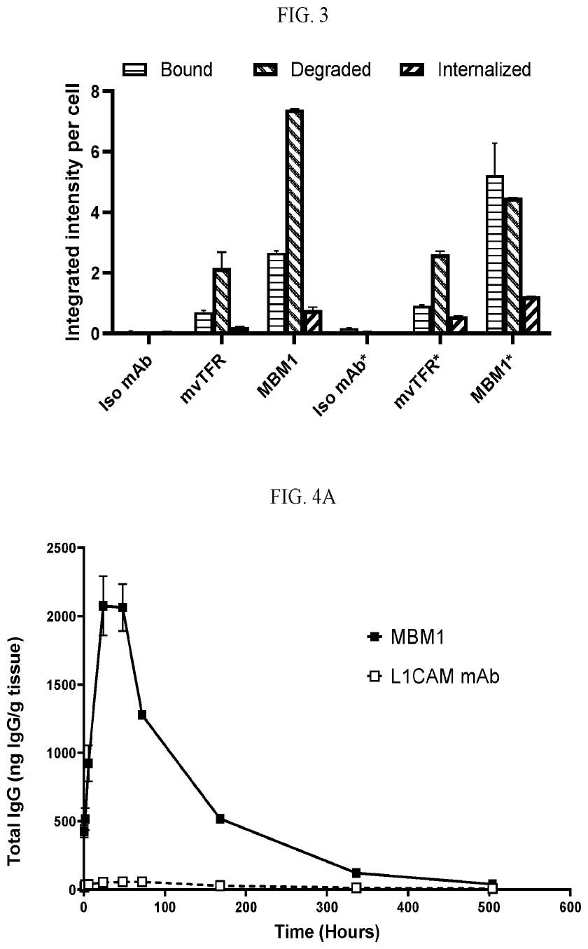

The following day, cells are washed, incubated for 20 min with NucBlue Hoechst dye (Thermo Fisher #R37605), washed again then imaged with Cytation 5 high content imager (Biotek). DyLight 650 signal measures total antibody levels, DyLight 650 plus BHQ3 signal measures degradation signal that increases DyLight 650 fluorescence when BHQ3 dye is liberated and FRET quenching is lost, while pHAb pH sensor dye signal measures only internalized fluorescence. Excess goat gamma globulin is added to reduce non-specific binding and uptake of antibodies into the cells. The intensity of the signal in each well is divided by the number of Hoechst-stained nuclei to determine signal intensity per cell. Wells are analyzed in duplicates, and for each well, approximately 20 k cells are analyzed from images taken with a 4× objective. The background signal is determined from human IgG isotype control and subtracted from the final value. Results are provided in FIG. 3.

High content imaging data demonstrates cellular activity (binding, internalization and degradation properties) of exemplified multispecific binding molecule is greater than that of monovalent TFR antibody and isotype control in neurons. In addition, when conjugated to siRNA the activity of exemplified multispecific binding molecule is not substantially altered. Isotype control and isotype control conjugated to siRNA lacked activity, indicating the activity shown for exemplified multispecific binding molecule is target receptor specific.

### In Vitro Potency Assessment in Murine Cortical Neurons

Murine primary cortical neurons are isolated from wild type C57BL6 mouse embryos at E18. Cells are plated in poly-D-lysine coated 96-well plates at a density of 40 k cells/well and cultured in NbActiv1 (BrainBits, LLC) containing 1% Antibiotic/Antimycotic (Corning) for 7 days at 37° C. in a tissue culture incubator in a humidified chamber with 5% C02. On day 7, half the medium is removed from each well and 2× concentration of one of: (i) siRNA (targeting a desired therapeutic target within the CNS); (ii) control siRNA (naked siRNA not targeting a therapeutic target within the CNS); (iii) hIgG4 isotype control antibody having an siRNA linked at HC Constant region 1 (Iso mAb*), (iv) monovalent TfR antibody having a cholesterol conjugated siRNA at HC Constant region 1 (mvTFR*), or (v) MBM1 (of Table 2) having a cholesterol conjugated siRNA at HC Constant region 1 (MBM1*), in culture media with 2% FBS is added for treatment at a range of siRNA concentrations and incubated with cells for additional 7 days. At the end of siRNA treatment, RT-qPCR is performed to quantify targeted mRNA levels using TaqMan Fast Advanced Cell-to-CT kit. Specifically, cells are lysed, cDNA is generated on Mastercycler X50a (Eppendorf), and qPCR is carried out on QuantStudio 7 Flex Real-Time PCR System (Applied Biosystems). Gene expression levels of the therapeutic target are normalized by β-actin (ThermoFisher Mm02619580_g1) using respective probes. Results are provided in Table 4:

TABLE 4
In Vitro Potency in Murine Cortical Neurons

| | siRNA | control siRNA | | | |
|---|---|---|---|---|---|
| | (targeting | (not targeting | | | |
| | therapeutic | therapeutic | Iso | | |
| | target) | target) | mAb* | mvTFR* | MBM1* |
| IC50 | 0.2 | 1.8 | 3.1 | 0.083 | 0.007 |
| (nM) | | | | | |

Results provided in Table 4 demonstrate exemplified multispecific binding molecule provides potency for linked siRNA targeting a therapeutic target multiple order of magnitudes greater than unconjugated (e.g., naked siRNA) and greater than an order of magnitude greater than siRNA targeting a therapeutic target linked only to mvTFR.

### Brain Pharmacokinetic Assessment

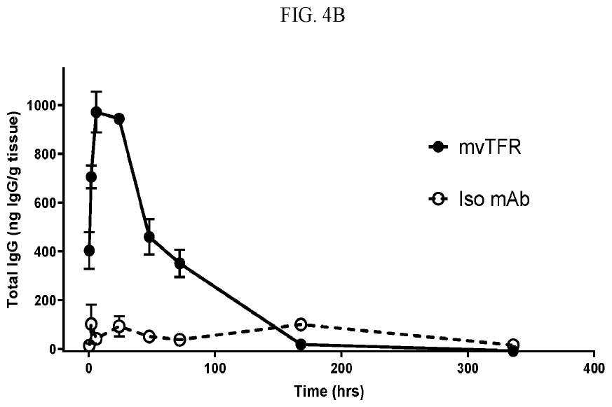

Brain pharmacokinetics of exemplified multispecific binding molecules of the present disclosure may be assessed according to the following. Briefly, male FVB mice at 8 weeks are dosed with (i) Iso mAb, (ii) mvTFR, (iii) L1CAM antibody (L1CAM mAb) and (iv) MBM1 at 5 mg/kg, sacrificed and perfused at time points: 0.5, 2, 6, 24, 48, 72, 168, 336, and 504 hours (n=3 per time point). Levels of the respective molecules in the brain is determined by ELISA (as assessed by area under the curve (AUC)). Following procedures as described herein, Iso mAb and L1CAM mAb demonstrate low levels in the brain. mvTFR achieves peak Cmax of around 1000 ng/g between 24 and 48 hrs and demonstrates brain accumulation which clears by 168 hours. MBM1 demonstrates a higher Cmax than mvTFR between 24 and 48 hr and demonstrates delayed clearance from the brain with 500 ng/ml detectable at 168 hr, and over 100 ug detected at 336 hrs. Results are provided in FIG. 4A and FIG. 4B.

Results suggest the combination of blood-brain barrier target binding domain and CNS Target (neuron) binding domain demonstrate enhanced molecule brain PK over mvTFR or L1CAM targeting molecules alone.

### In Vivo Pharmacodynamic Assessment

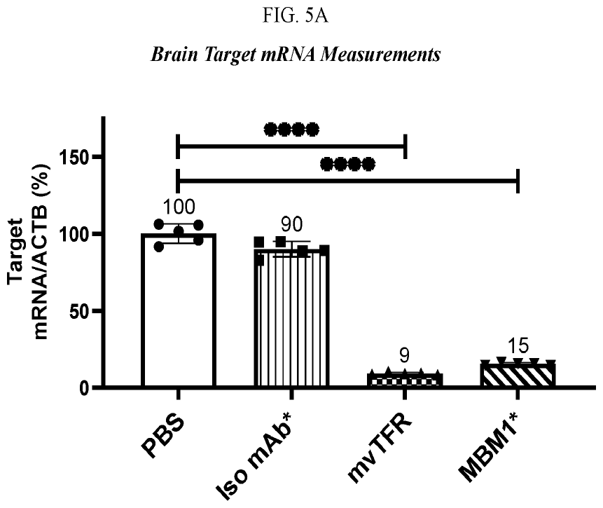

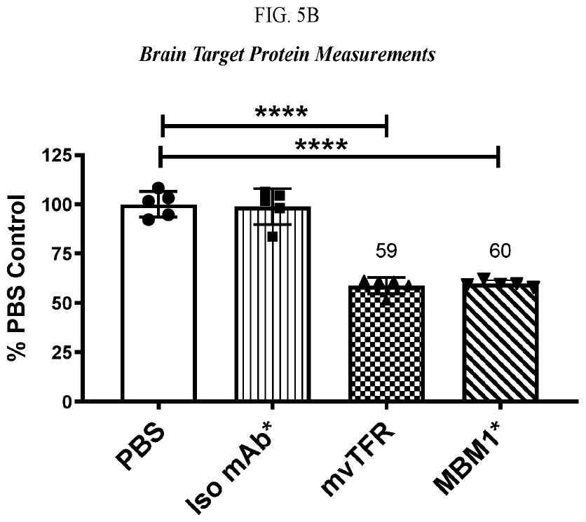

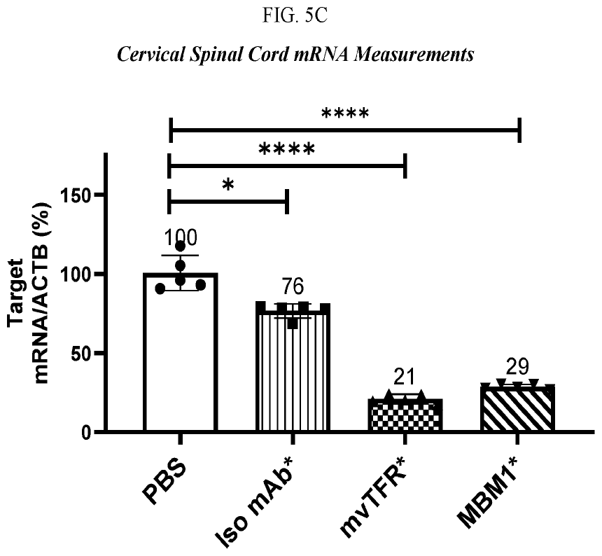

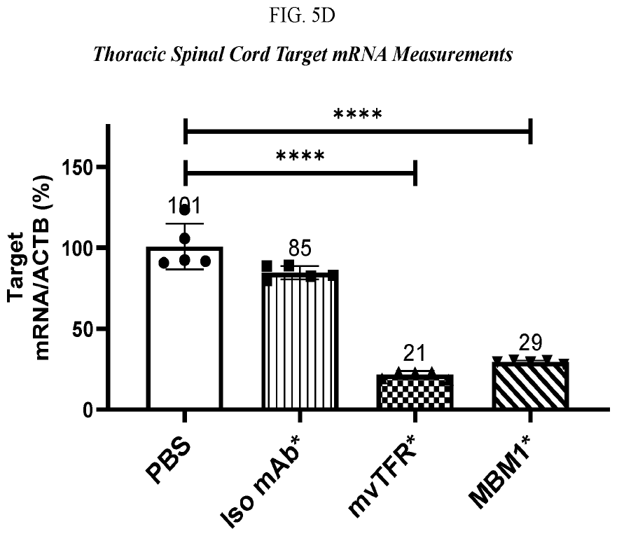

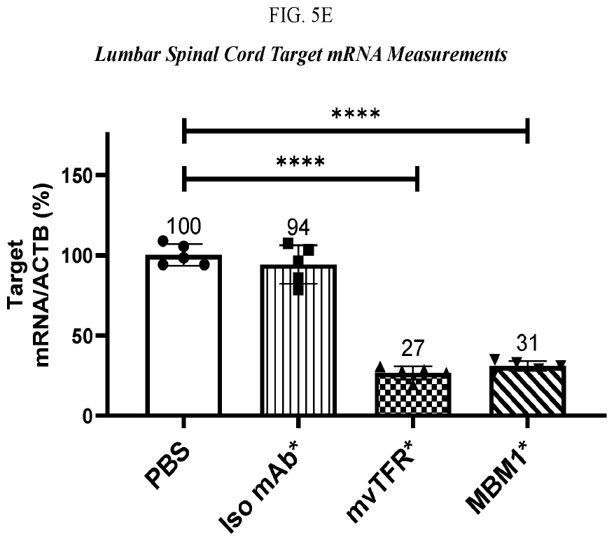

Pharmacodynamic properties of exemplified multispecific binding molecules of the present disclosure may be assessed according to the following. Briefly, Iso mAb*, mvTFR* and MBM1* are dosed in 8-week-old FVB mice at 10 mg/kg effective siRNA concentration intravenously once a week for 4 weeks. In addition, mouse anti-CD4 antibody (GK1.5) is dosed at 10 mg/kg to ablate CD4 positive T cells to mitigate undesired pharmacokinetic consequences resulting from spurious anti-drug antibody responses to injected compounds. Mice are sacrificed 168 hr after final dose and perfused to collect brain and spinal cord to assess target mRNA levels by RT-qPCR and target protein levels by ELISA in tissue homogenates. Results are provided in FIG. 5A-5E.

Results demonstrate significant improvement in mRNA and protein levels over PBS and Iso mAb (NOTE: is it believed that MBM1* reductions would be further enhanced in higher L1CAM populations).

In vivo Assessment of Brain Accumulation, Peripheral Tissue Accumulation and CSF and Plasma Accumulation

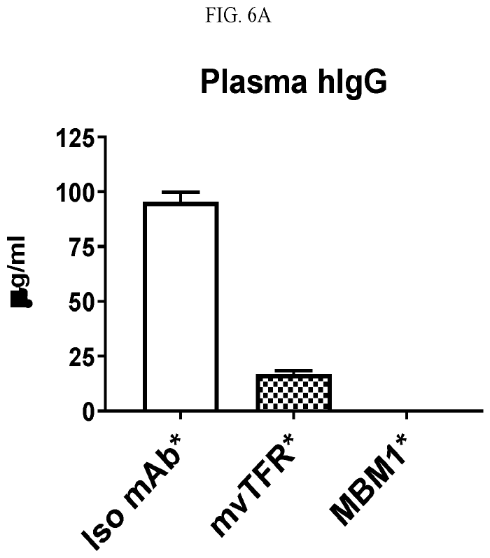

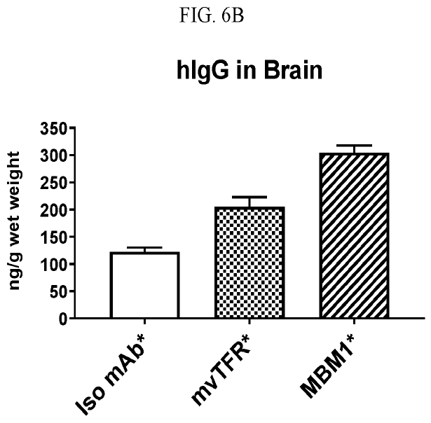

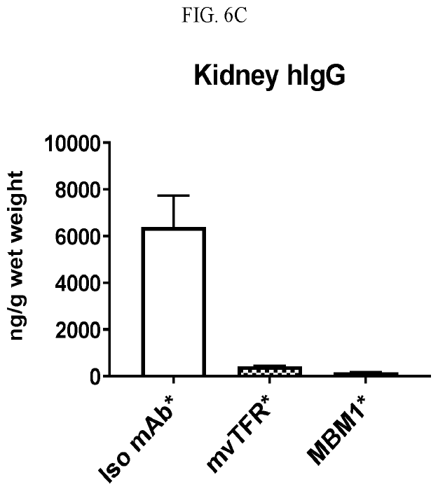

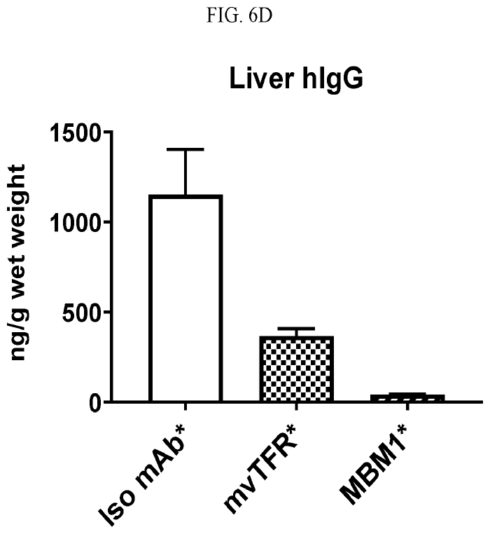

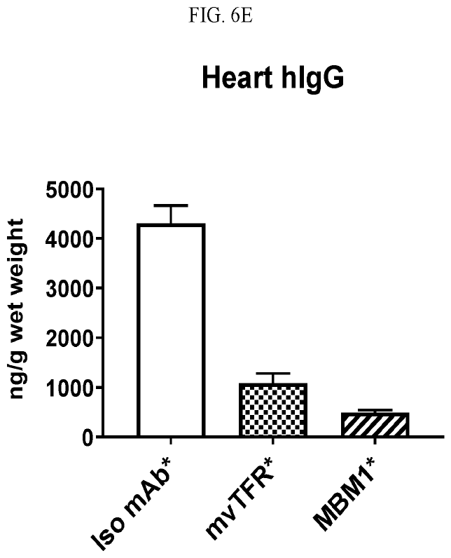

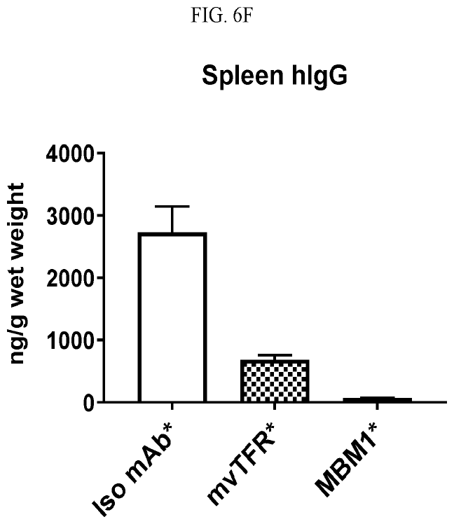

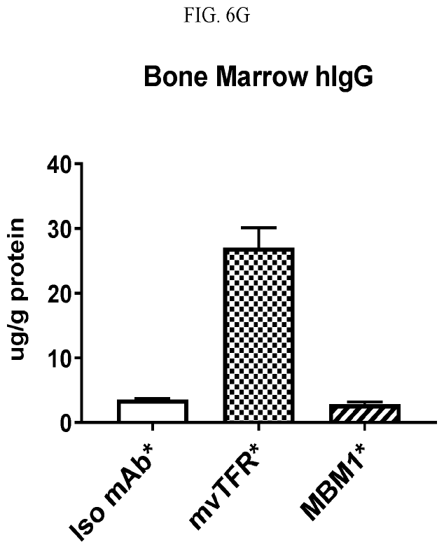

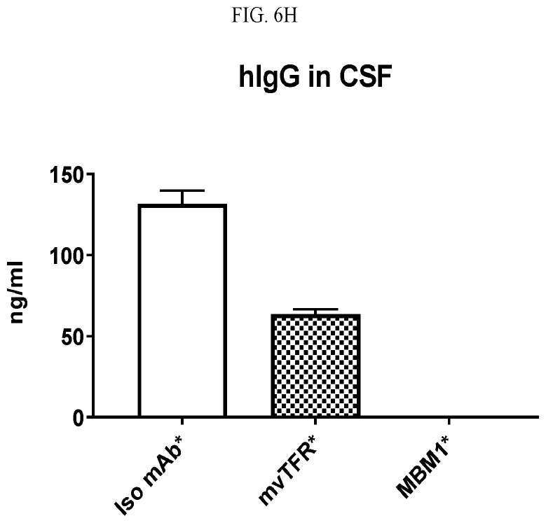

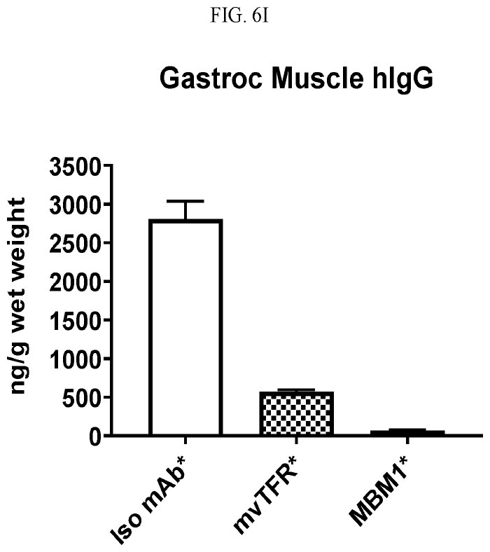

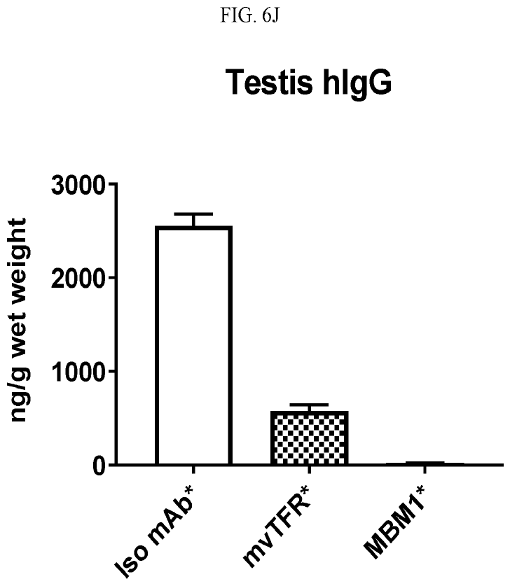

Brain and peripheral accumulation of exemplified multispecific binding molecules of the present disclosure may be assessed according to the following. Briefly, following a pharmacodynamic study as described above, brain tissue, CSF, plasma and peripheral tissue are collected and levels of respective administered molecules are determined by ELISA. Results are provided in FIGS. 6A thru 6J.

Results demonstrate levels of MBM1* in the brain were approx. 200% greater than Iso mAb* and approx. 100% higher than mvTFR*, Results also demonstrate that only MBM1* levels are undetectable in plasma, CSF and all peripheral organs (bone marrow, gastroc muscles, liver, kidney, heart, spleen and testis). In addition, MBM1* demonstrated levels in bone marrow equivalent to Iso mAb* and less than 10% mvTFR*. The results demonstrate MBM1* achieves increased brain presence, but not peripheral accumulation including bone marrow accumulation.

Sequences

SEQ ID NO. 1 - Exemplified Anti-L1CAM HC
EVQLVESGGGVVQPGRSLRLSCAASGFTFSRFGMHWVRQAPGKGLEWVAFISNDGS
NKYYADSVKGRFTISRDNSKNTLYLQMNSLRPEDTAVYYCARGRAYGSGSLFDPWG
QGTLVTVSSASTKGPCVFPLAPCSRSTSESTAALGCLVKDYFPEPVTVSWNSGALTSG
VHTFPAVLQSSGLYSLSSVVTVPSSSLGTKTYTCNVDHKPSNTKVDKRVESKYGPPC
PPCPAPEAAGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPV
LDSDGSFLLYSKLTVDKSRWQEGNVFSCSVMHEALHNHYTQKSLSLSLG
SEQ ID NO. 2 - Exemplified Anti-L1CAM LC
DIQMTQSPSSLSASVGDRVTITCKASQGISRFLSWFQQKPGKAPKSLIYAVSSLVDGV
PSRFSGSGSGTDFTLTISSLQPEDFATYYCVQYNSYPYGFGGGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO. 3 - Exemplified Anti-TFR HC
QSLEESGGDLVKPEGSLTLTCTASGFSFSGSYWICWVRQAPGKGLEWIGCIYSTSGGR
TYYASWVKGRFTISKTSSTTVTLQMTSLTAADTATYFCARGDDSISDAYFDLWGPGT
LVTVSSASTKGPCVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEAAGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG
SEQ ID NO. 4 - Exemplified Anti-TFR LC
ALDMTQTASPVSAAVGGTVTINCQSSQSVYNNNRLAWYQQKPGQPPKLLIYDASTL
ASGVPSRFKGSGSGTQFTLTISGVQSDDSATYYCQGTYFSSGWSWAFGGGTEVVVK
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO. 5 - Exemplified Anti-TFR HCVR
QSVEESGGRLVTPGTPLTLTCTVSGFSLSTYAMIWVRQAPGKGLEYIGFIDNDYKAFY
ATWTNGRFTVSRTSTTVDLKMTSLTTEDTATYFCARYGSSSASDLWGQGTLVTVSS
SEQ ID NO. 6 - Exemplified Anti-TFR LCVR
ALVMTQTPASVSAAVGDTVTIKCQASESIITWLAWYQQRPGQPPKLLIYRASTLASG
VPSRFKGSGSGTQFTLTISDLEAADAAIYYCQNNAGDSSYGFAFGGGTEVVVK
SEQ ID NO. 7 - Exemplified Anti-TFR HCVR
QSVEESGGRLVTPGTPLTLTCTVSGIDLSGSAMSWVRQAPGKGLEWIGIIYARGGTY
YATWAQGRFTISKTSTTVDLKITSPTIEDTATYFCSRGYTDGFDLWGQGTLVTVSS
SEQ ID NO. 8 - Exemplified Anti-TFR LCVR
ALVMTQTPSPVSAAVGGTVSINCQSTKSIYNNKYLSWYQQKPGQPPKLLIYDASDLA
SGVPSRFKGSGSGTQFTLTISGVQADDAATYYCLGGYSSDSENAFGGGTEVVVK
SEQ ID NO. 9 - Exemplified Anti-TFR HCVR
ALVMTQTPASVSEPVGGTVTIKCQASQSISSWLAWYQQKPGQPPKLLIYRASTLASG
VSSRFKGSGSGTDFTLTISDLEAADAATYYCQTSGAMGTYGGAFGGGTEVVVK
SEQ ID NO. 10 - Exemplified Anti-TFR LCVR
QSVEESGGRLVTPGTPLTLTCTASGFSLSSYYMSWVRQAPGKGLEWIGFIYTDGSTY
YASWAKGRFTISKTSTTVDLKITSPTTEDTATYFCARYSGSGLDLWGLGTLVTVSS
SEQ ID NO. 11 - Exemplified Anti-CD98 HCVR
QSLEESGGDLVKPGASMTLTCTASGFSFSSGYWICWVRQAPGKGLEWIACIHSVRSH
MTYYASWAKGRFTISKTSSTTVTLQMTSLTAAATATYFCARDASGVWNYFTLWGP
GTLVTVSS
SEQ ID NO. 12 - Exemplified Anti-CD98 LCVR
AEVVMTQTPSSVSAAVGGTVTIKCQASQNINSWLSWYQQKPGQRPKLLIYSASTLAS
GVPSRFEGSGSGTEYTLTISDLECDDAATYYCQSSYGSSYDFGGGTEVVVK
SEQ ID NO. 13 - Exemplified Anti-CD98 HCVR
QEQLVESGGGLVQPEGALTLTCTASGLDFSSSYWICWVRQAPGKGLEWIACVHAGS
GGYNYYATWAKGRFTISRTSSTTVTLQMTSLTAADTATYFCARGVFPDYVDATLFN
LWGPGTLVTVSS
SEQ ID NO. 14 - Exemplified Anti-CD98 LCVR
AAVMTQTASPVSAAVGGTVTINCQASQSVSSAYLSWYQQKPGQPPKLLIYKASTLAS
GVSSRFKGSGSGTEYTLTISGVQCDDAATYYCLYGDYSGRSNAFGGGTEVVVK
SEQ ID NO. 15 - Exemplified Anti-CD98 HCVR
QEHMEESGGDLVKPEGSLTLTCTASGFSFSRMYWICWVRQAPGKGLEWIACIYTGD
GNTYYASWAKGRFTISKTSSSTVTLQMTSLTAADTATYFCARDPDGYSIYYFNLWGP
GTLVTVSS
SEQ ID NO. 16 - Exemplified Anti-CD98 LCVR
ALVMTQTPASVSTAVGGTVTISCQASQSINSWLAWYQQKPGQRPNLLIYGASKLPSG
VPSRFKGSGSGTEFTLTISDLECSDAATYYCAGYKTYSNDDNAFGGGTEVVVK
SEQ ID NO. 17 - Exemplified Anti-CD98 HCVR
QQLVESGGGLVQPGASLTLTCTASGFSFSSSYWICWVRQAPGKGLEWSACIYGGIPY
YASWAKGRFTISKTSSTTVTLQMTSLTVADTATYFCARDDYYKSAWGGYNLWGPG
TLVTVSS
SEQ ID NO. 18 - Exemplified Anti-CD98 LCVR
AAVMTQTPSPVSAAVGGTVTISCQASQSVYGNNYFAWFQQKPGQPPKLLIYKASTL
ASGVPSRFKGSGSGTQFTLTISGVECDDAATYYCAGYKSYSNDGYAFGGGTEVVVK
SEQ ID NO. 19 - Exemplified Anti-L1CAM HCVR
EVQLVESGGGVVQPGRSLRLSCAASGFTFSRFGMHWVRQAPGKGLEWVAFISNDGS
NKYYADSVKGRFTISRDNSKNTLYLQMNSLRPEDTAVYYCARGRAYGSGSLFDPWG
QGTLVTVSS
SEQ ID NO. 20 - Exemplified Anti-L1CAM LCVR
DIQMTQSPSSLSASVGDRVTITCKASQGISRFLSWFQQKPGKAPKSLIYAVSSLVDGV
PSRFSGSGSGTDFTLTISSLQPEDFATYYCVQYNSYPYGFGGGTKVEIK
SEQ ID NO. 21 - Exemplified Anti-TFR HCVR
QSLEESGGDLVKPEGSLTLTCTASGFSFSGSYWICWVRQAPGKGLEWIGCIYSTSGGR
TYYASWVKGRFTISKTSSTTVTLQMTSLTAADTATYFCARGDDSISDAYFDLWGPGT
LVTVSS
SEQ ID NO. 22 - Exemplified Anti-TFR LCVR
ALDMTQTASPVSAAVGGTVTINCQSSQSVYNNNRLAWYQQKPGQPPKLLIYDASTL
ASGVPSRFKGSGSGTQFTLTISGVQSDDSATYYCQGTYFSSGWSWAFGGGTEVVVK

1. A multispecific binding molecule comprising:
a first binding domain that specifically binds a blood brain barrier target; and
a second binding domain that specifically binds a neuron target
wherein the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A,
wherein the neuron target is one of L1CAM, THY1, KIT, GRIA1, GRM2, TMEM130, NSG1, BASP1, NSTR1, SLC22A17, NCAM1, NCAM2, SLC6A17, SCN3B and P2Y12R,
wherein the multispecific binding molecule is a heterodimeric immunoglobulin G antibody.

2. A multispecific binding molecule comprising:
a first binding domain that specifically binds a blood brain barrier target; and
a second binding domain that specifically binds an astrocyte target,
wherein the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A,
wherein the astrocyte target is one of CD49f, AQP4, GLT1, GLAST, A2AR, TGFbR, S1P1, IL1R, IL6R, IL17R and IFNgR,
wherein the multispecific binding molecule is a heterodimeric immunoglobulin G antibody.

3. A multispecific binding molecule comprising:
a first binding domain that specifically binds a blood brain barrier target; and
a second binding domain that specifically binds a microglia target,
wherein the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A,
wherein the microglia target is one of TREM2, TMEM119, CD11b, CD14, CD16, CD32, CD33, CD40, CD45, CD64, CD68, CD80, CD115, CD172a, CX3CR1, FCER1G, F4/80, FCRLS, Siglec, Glut5, and P2Y12,
wherein the multispecific binding molecule is a heterodimeric immunoglobulin G antibody.

4. A multispecific binding molecule comprising:
a first binding domain that specifically binds a blood brain barrier target; and
a second binding domain that specifically binds a neuron target,
wherein the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A,
wherein the neuron target is one of L1CAM, THY1, KIT, GRIA1, GRM2, TMEM130, NSG1, BASP1, NSTR1, SLC22A17, NCAM1, NCAM2, SLC6A17, SCN3B and P2Y12R,
wherein the first binding domain is a Fab, scFv, Fv, or scFab,
wherein the second binding domain is a Fab, scFv, Fv, or scFab.

5. A multispecific binding molecule comprising:
a first binding domain that specifically binds a blood brain barrier target; and
a second binding domain that specifically binds an astrocyte target,
wherein the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A,
wherein the astrocyte target is one of CD49f, AQP4, GLT1, GLAST, A2AR, TGFbR, SlP1, IL1R, IL6R, IL17R and IFNgR,
wherein the first binding domain is a Fab, scFv, Fv, or scFab,
wherein the second binding domain is a Fab, scFv, Fv, or scFab.

6. The multispecific binding molecule of claim 4, wherein the first binding domain and the second binding domain are linked by one of a thiol-based reactive group, an amine-based reactive group, aldehyde-reactive groups or carbohydrate-based groups.

7. The multispecific binding molecule of claim 4, wherein the first binding domain and the second binding domain are linked by one of a PEG-linker, an amino acid linker, or a glycan linker.

8. The multispecific binding molecule of claim 1, wherein the blood brain barrier target is TFR and the neuron target is L1CAM.

9. The multispecific binding molecule of claim 1, wherein a therapeutic composition is linked to the multispecific binding molecule.

10. The multispecific binding molecule of claim 9, wherein the therapeutic composition is one of a peptide, antibody or fragment thereof, and an oligonucleotide.

11. The multispecific binding molecule of claim 10, wherein the therapeutic composition is an oligonucleotide targeting one of MAPT, SNCA, APP, BAC1, ATXN2, ATXN3, SARM1, APOE, FMR1, LRRK2, HTT, SOD1, SCN10A, SCN9A and CACNA1B.

12. The multispecific binding molecule of claim 10, wherein the therapeutic composition is an antibody or fragment thereof that binds one of amyloid beta, BACE1, MAPT, SNCA, APP, BAC1, ATXN2, ATXN3, SARM1, APOE, FMR1, LRRK2, HTT, SOD1, SCN10A, SCN9A and CACNA1B.

13. A method of delivering a therapeutic composition to the central nervous system of a mammal comprising administering the therapeutic composition to the mammal,
wherein the therapeutic composition is linked to a multispecific binding molecule of claim 1, wherein said step of administering comprises intravenous, subcutaneous, intramuscular or interparental administration of the multispecific binding molecule, and
wherein therapeutic composition accumulation in peripheral tissue of the mammal is reduced compared to when the therapeutic composition is not linked to the multispecific binding molecule.

14. The method of claim 13, wherein accumulation of therapeutic composition in peripheral tissue is assessed at least 24 hours, 48 hours, 72 hours, 96 hours, 120 hours, or 144 hours after said step of administering.

15. The method of claim 13, wherein the peripheral tissue is selected from one of bone marrow, liver, plasma, testis, muscle, or kidney.

16. The method of claim 13, wherein said step of administering comprises intravenous, subcutaneous, intramuscular or interparental administration of the multispecific binding molecule.

17. (canceled)

18. A multispecific binding molecule comprising:
a first binding domain that specifically binds a blood brain barrier target; and
a second binding domain that specifically binds a glial cell target,
wherein the blood brain barrier target is one of TFR, low density lipoprotein receptor-related protein 1 (LRP1), LRP2 or megalin/GP330, LRP3, LRP5, LRP6, LRP8, insulin-like growth factor (IGF), Transferrin Receptor-1, CD98 heavy chain (SLC3A2), large amino acid transporter small subunit 1 (LAT-1), insulin receptor, low-density lipoprotein (LDL) receptor, CDC50A,
wherein the second binding domain specifically binds a microglia target, wherein the microglia target is one of TREM2, TMEM119, CD11b, CD14, CD16, CD32, CD33, CD40, CD45, CD64, CD68, CD80, CD115, CD172a, CX3CR1, FCER1G, F4/80, FCRLS, Siglec, Glut5, and P2Y12
wherein the first binding domain is a Fab, scFv, Fv, or scFab,
wherein the second binding domain is a Fab, scFv, Fv, or scFab.

19. A method of delivering a therapeutic composition to the central nervous system of a mammal comprising administering the therapeutic composition to the mammal, wherein the therapeutic composition is linked to a multispecific binding molecule of claim 2, wherein said step of administering comprises intravenous, subcutaneous, intramuscular or interparental administration of the multispecific binding molecule, and wherein therapeutic composition accumulation in peripheral tissue of the mammal is reduced compared to when the therapeutic composition is not linked to the multispecific binding molecule.

20. A method of delivering a therapeutic composition to the central nervous system of a mammal comprising administering the therapeutic composition to the mammal, wherein the therapeutic composition is linked to a multispecific binding molecule of claim 3, wherein said step of administering comprises intravenous, subcutaneous, intramuscular or interparental administration of the multispecific binding molecule, and wherein therapeutic composition accumulation in peripheral tissue of the mammal is reduced compared to when the therapeutic composition is not linked to the multispecific binding molecule.
